(19) United States
(12) Patent Application Publication
(10) Pub. No.: US 2008/0280983 A1
(43) Pub. Date: Nov. 13, 2008

(54) METHODS OF TREATING RETT SYNDROME

(75) Inventors: Ranga Krishnan, Chapel Hill, NC (US); Atul Pande, Cary, NC (US)

Correspondence Address: KNOBBE MARTENS OLSON & BEAR LLP, 2040 MAIN STREET, FOURTEENTH FLOOR, IRVINE, CA, 92614, US

(73) Assignee: CeNeRx BioPharma, Cary, NC (US)

(21) Appl. No.: 12/117,586

(22) Filed: May 8, 2008

Related U.S. Application Data
(60) Provisional application No. 60/917,019, filed on May 9, 2007.

(51) Int. Cl. A61K 31/197; A61P 25/00

(52) U.S. Cl. 514/563

(57) ABSTRACT

The present embodiments relate to methods for the treatment of Rett Syndrome. More specifically, the present embodiments relate to pivagabine for use in the treatment of Rett Syndrome. Some embodiments relate to treatment of a patient with Rett Syndrome. Other embodiments relate to treatment of the indications of Rett Syndrome.

### CROSS-REFERENCE TO RELATED APPLICATIONS

This application claims the benefit of U.S. Provisional Application No. 60/917,019 filed May 9, 2007, the disclosure of which is herein incorporated by reference in its entirety.

### BACKGROUND OF THE INVENTION

1. Field of the Invention

The present embodiments relate to methods for the treatment of Rett Syndrome. More specifically, the present embodiments relate to pivagabine for use in the treatment of Rett Syndrome.

2. Description of the Related Art

Rett Syndrome (RTT) is a debilitating neurological disorder diagnosed almost exclusively in females. Females with RTT appear to develop normally until 6 to 18 months of age when they enter a period of regression, losing speech and motor skills. Most develop repetitive hand movements, irregular breathing patterns, seizures and extreme motor control problems. RTT leaves its victims profoundly disabled, requiring maximum assistance with every aspect of daily living.

Rett is the most physically disabling of the family of autism diseases. Girls are born healthy but then the culprit gene manifests, destroying speech and normal movement. A classic sign is hands that can do nothing but wring. Many children never walk; those that do have a stiff-legged gait. Symptoms usually appear between 6 and 18 months of age. Patients can live to adulthood, although many die of infections before then.

Historically, RTT was believed to affect 1 in 10,000 females. Many scientists now believe that the prevalence of RTT is in fact much higher. There are potentially thousands of girls and women undiagnosed or misdiagnosed (eg., autism, cerebral palsy). Although rare, it is possible for males to have RTT.

RTT knows no geographic, racial or social boundaries. Fewer then 1% of Rett cases are familial. Any expectant parent is at risk for having a child with RTT. Particular mutations of the MECP2 gene are thought to cause RTT. Over a hundred separate mutations in MECP2 have been identified to date. Although some individuals with RTT die at a young age, the majority live into adulthood.

Pivagabine, or 4-[(2,2-dimethyl-1-oxopropyl)amino]butanoic acid:

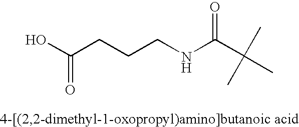

is a hydrophobic derivative of GABA which can cross the haemato-encephalic barrier and act as an active pharmaceutical compound against hypertension and cerebral disturbances, such as epilepsy. Pivagabine has been found to be substantially free of toxic effects in vivo in mouse and in rat; in fact, LD50 by intravenous route is 1750 mg/kg in the mouse while no toxic effects were observed up to the dose of 1 g/kg by intraperitoneal route in the rat. It shows a certain anti-depressive and anxiolytic activity in the mouse and can be used to treat mood disturbances, anxiety disorders, somatoform disorders and adjustment disorders.

However, though pivagabine was originally developed as an analog of GABA with the proposition that it would be hydrolyzed to GABA after entering the brain, pivagabine does not appear to bind to GABA receptors. Therefore, its mechanism of action is likely not related to GABA. In attempts to elucidate the mechanism of action it was discovered that pivagabine affects the corticotrophin-releasing hormone (CRH) content of specific brain regions (hypothalamus and cerebral cortex) particularly during stress conditions (application of foot shock in rodents). This effect is centrally mediated and does not require an intact hypothalamic-pituitary-adrenal axis. Also, in modulating CRH, pivagabine is not acting as a CRF-1 receptor antagonist since it does not reverse the effects of CRH administered directly into the brain of rodents.

### SUMMARY OF THE INVENTION

One embodiment relates to a method of treating a patient with symptoms of Rett Syndrome, comprising administering an amount of pivagabine to the patient effective to reduce said symptoms.

In one aspect of the embodiment, the patient is a mammal.

In another aspect of the embodiment, the patient is a human.

In one aspect of the embodiment, the symptoms are selected from anxiety, ataxia, stressful reactions to novel situations, convulsions, compulsive or repetitive behavior, chorea, memory impairment, social withdrawal, and mental retardation.

In one aspect of the embodiment, the pivagabine is administered by one or more of the routes consisting of intravenous, intraperitoneal, inhalation, intramuscular, subcutaneous and oral.

In another aspect of the embodiment, the pivagabine is administered orally.

In one aspect of the embodiment, the pivagabine has the following structure:

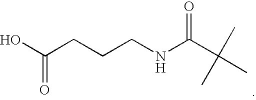

Another embodiment relates to a method of treating the indications of Rett Syndrome in a patient comprising administering to said patient a therapeutically effective amount of pivagabine.

In one aspect of the embodiment, the patient is a mammal.

In another aspect of the embodiment, the patient is a human.

In one aspect of the embodiment, the pivagabine is administered by one or more of the routes consisting of intravenous, intraperitoneal, inhalation, intramuscular, subcutaneous and oral.

In one aspect of the embodiment, the pivagabine is administered orally.

In another aspect of the embodiment, the pivagabine has the following structure:

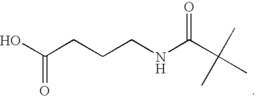

Yet another embodiment relates to a method of treating or preventing the symptoms of Rett Syndrome in a mammal, comprising the step of administering to said mammal a therapeutically effective amount of pivagabine and a pharmaceutically acceptable vehicle.

In one aspect of the embodiment, the mammal is human.

In one aspect of the embodiment, the symptoms are selected from anxiety, ataxia, stressful reactions to novel situations, convulsions, compulsive or repetitive behavior, chorea, memory impairment, social withdrawal, and mental retardation.

In one aspect of the embodiment, the pivagabine is administered by one or more of the routes consisting of intravenous, intraperitoneal, inhalation, intramuscular, subcutaneous and oral.

In another aspect of the embodiment, the pivagabine is administered orally.

In one aspect of the embodiment, the pivagabine has the following structure:

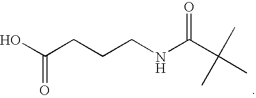

Another embodiments relates to a method of treating Rett Syndrome, comprising administering pivagabine.

In one aspect of the embodiment, the pivagabine is administered by one or more of the routes consisting of intravenous, intraperitoneal, inhalation, intramuscular, subcutaneous and oral.

In one aspect of the embodiment, the pivagabine is administered orally.

In one aspect of the embodiment, the pivagabine has the following structure:

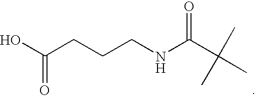

### BRIEF DESCRIPTION OF THE DRAWINGS

FIG. 1 shows the activity of pivagabine against electroshock-induced convulsions in rats.

FIG. 2 shows the LD₅₀ of strychnine obtained in controls and pivagabine- and mephenesine-pretreated animals.

FIG. 3 shows the anticonvulsive activity of pivagabine on pentetrazol-induced convulsions in rats.

FIG. 4 shows the anticonvulsive activity of pivagabine on bicucullin-induced convulsions in rats.

FIG. 5 shows the dose-effect relationship of pivagabine administered 1 hour prior to isoniazide.

FIG. 6A shows the effect of pivagabine on ambulation measured by number of sectors explored per unit time.

FIG. 6B shows the effect of pivagabine on the number of rearing movements.

FIG. 6C shows the effect of pivagabine on defecation as measured by number of fecal boluses produced.

FIG. 7A shows the effect of pivagabine on the time taken to reach exit as a function of successive days.

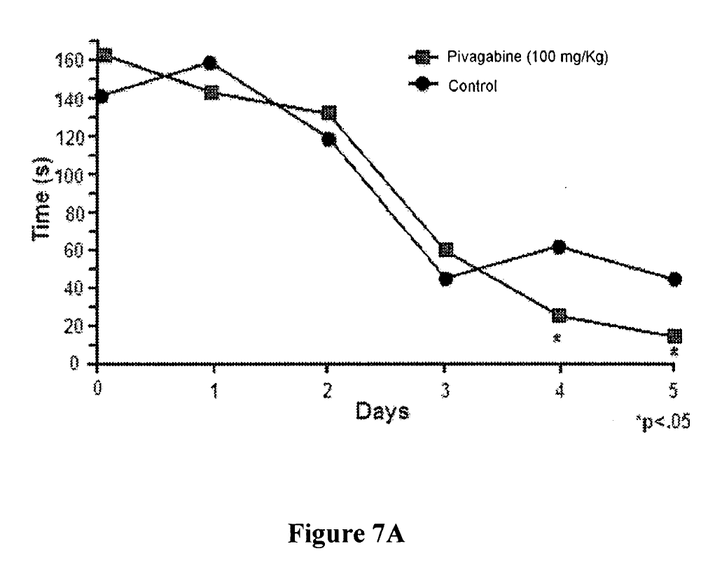

FIG. 7B shows the effect of pivagabine on the number of errors made on each day of testing.

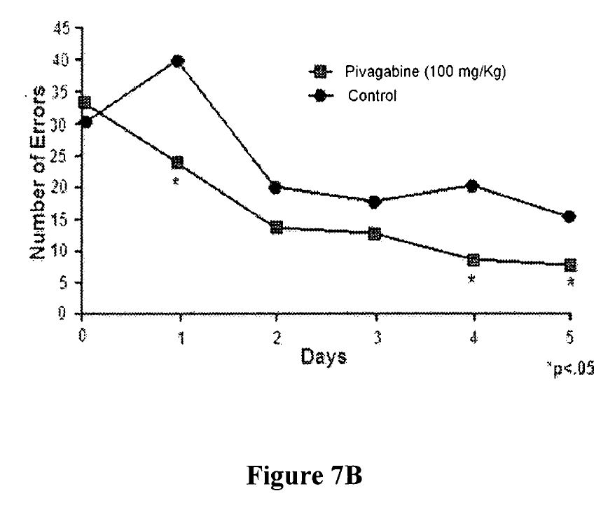

FIG. 8A shows the effect of pivagabine on performance on the Rota-Rod Test.

FIG. 8B shows the effect of pivagabine on the Rota-Rod Test when controlled for baseline bodyweight.

FIG. 9A shows the effect of pivagabine on the time to reach the end in the Taut Thread Test.

FIG. 9B shows the effect of pivagabine on number of resting positions in the Taut Thread Test. Right.

FIG. 10 shows the effect of pivagabine on foot-shock stress-induced change in GABA_{A} receptor binding.

FIG. 11 shows the time-course of the effect of pivagabine on TBPS binding.

FIG. 12A shows the effect of pivagabine on CRH levels in the hypothalamus induced by foot shock stress.

FIG. 12B shows the effect of pivagabine on CRH levels in the cerebral cortex induced by foot shock stress.

### DETAILED DESCRIPTION

The present embodiments are related to treatments for RTT. More specifically, some embodiments relate to treating RTT with the administration of pivagabine. Even more specifically, some embodiments relate to methods of treating RTT by administering an amount of pivagabine to the body of a patient effective to reduce or prevent the symptoms of RTT. Other embodiments relate to the administration of pivagabine in order to reduce the symptoms of RTT, such as, for example anxiety, ataxia, stressful reactions to novel situations, convulsions, compulsive or repetitive behavior, chorea, memory impairment, social withdrawal, mental retardation, and autism.

Rett syndrome (RTT), a postnatal neurodevelopmental disorder, is caused by mutations in the methyl-CpG-binding protein 2 (MECP2) gene. Children with RTT display cognitive and motor abnormalities as well as autistic features. Some embodiments relate to the discovery that mice bearing a truncated MECP2 allele (MECP2^{308/Y} mice) display increased anxiety-like behavior and an abnormal stress response as evidenced by elevated serum corticosterone levels. These mice display increased corticotropin-releasing hormone (CRH) gene expression in the paraventricular nucleus of the hypothalamus, the central amygdala, and the bed nucleus of the stria terminalis. Other embodiments relate to the discovery that MECP2 binds the CRH promoter, which is enriched for methylated CpG dinucleotides and the finding that the MECP230B protein was not detected at the CRH promoter. Some embodiments relate to CRH as a target of MECP2 and CRH overexpression in the development of specific symptoms of the Mecp2JV8IY mouse and patients suffering from RTT.

As described in Shahbazian M. et al., (2002) Neuron 35:243-254, herein incorporated by reference in its entirety, the MECP23iJR mouse carries a hypomorphic MECP2 allele in which a truncating mutation is placed after amino acid 308. MECP2^{308/Y} male mice are viable up to 1 year of age and replicate many aspects of human RTT, including cognitive and motor impairments, seizures, and social behavior deficits. Behavioral analysis of MECP2^{308/Y} mice on a highly mixed background indicated that they have increased anxiety in the open field assay. Additionally, MECP2^{308/Y} mice are characterized by a tremor that becomes more severe with handling, suggesting that they are easily stressed.

Therefore, the above described mouse model is used as a suitable model for studying RTT. The above observations are consistent with findings from clinical investigations described in Mount RH et al., (2002) Child Psychol. Psychiatry 43:1099-1110 and Sansom D et al., (1993) Dev. Med. Child Neurol. 35:340-345, both of which are herein incorporated by reference in their entirety, indicating that episodes of heightened anxiety occur more frequently in RTT (up to 75% of cases) than in other forms of mental retardation. Anxiety is also reported in cases in which MECP2 mutations cause additional neurobehavioral phenotypes. These features of RTT, together with preliminary observations of the behavior of MECP2^{308/Y} mice, suggest that anxiety is an important component of the behavioral phenotype of RTT and that MECP2 regulates key molecule(s) relevant to this behavior. Behavioral and physiologic analyses on MECP2^{308/Y} mice along with studies on the effect of MECP2 mutation on the molecular pathway that contributes to anxiety related behaviors confirmed the role of MECP2 in anxiety.

CRH is a target of MECP2 and behavioral and physiologic phenotypes in MECP2^{308/Y} mice are consistent with enhanced CRH signaling. Behaviorally, CRH is involved in inducing anxiety. For example, intracerebroventricular delivery of CRH into the central nervous system of rodents is anxiogenic. Likewise, transgenic mice overexpressing CRH have enhanced anxiety. Conversely, pharmacologic antagonists of CRH receptor-1 signaling are anxiolytic, and CRH knockout mice have reduced anxiety.

Like the behavioral effects of CRH, the effects of CRH on the physiologic response to stress are well known. CRH produced in the paraventricular nucleus of the hypothalamus (PVN) initiates hypothalamic-pituitary-adrenal (HPA) axis activation and elicits glucocorticoid release from the adrenal cortex. Thus, exogenous administration of CRH in the PVN of rodents transiently increases circulating corticosterone, and transgenic overexpression of CRH chronically elevates serum glucocorticoid levels. Conversely, CRH antagonists block glucocorticoid release, and both CRH and CRH receptor-1 knockout mice have blunted post stress serum glucocorticoid concentrations. Mecp2^{308/Y} mice display elevated PVN stress-induced serum corticosterone levels and increased PVN CRH expression relative to controls.

Increased anxiety-like behavior and HPA axis hyperactivity are not limited to Mecp2^{308/Y} mice but seem to be features that are shared by other mouse models of RTT as well as humans with RTT. As described in Gemelli et al., (2006) Biol. Psychiatry 59:468-476, herein incorporated by reference in its entirety, conditional Mecp2 knockout mice demonstrate increased anxiety-like behavior, whereas, as described in Nuber et al., (2005) Hum Mol. Genetics 14:2247-2256, herein incorporated by reference in its entirety, constitutive Mecp2-null mice demonstrate a trend toward increased serum glucocorticoid levels and have enhanced expression of two glucocorticoid-inducible genes, Sgk and Fkbp5. With regard to humans with RTT, surveys indicate that anxiety-like behaviors occur frequently. Furthermore, there is evidence of increased urinary cortisol excretion in females with RTT, suggesting that humans with MECP2 mutations also experience elevated serum glucocorticoid levels.

Some of the chronic effects of CRH and coriticosterone resemble characteristic aspects of RTT. Specifically, decreased dendritic branching, reduced synaptic plasticity, and memory impairment are features observed in the context of repeated glucocorticoid exposure, exposure to high concentrations of CRH, and Mecp2/MECP2 mutation. These similarities suggest that an overabundance of CRH and/or glucocorticoids contribute to other aspects of the RTT phenotype beyond anxiety-like behaviors.

MeCP2 modulates the transcriptional activity of CRH in WT mice, but CRH transcriptional regulation is impaired in MECP2^{308/Y} mice. CpG methylation at the CRH promoter is unaltered in the MECP2^{308/Y} mice, suggesting that enhanced CRH expression in Mecp2^{308/Y} mice is due to a functional defect in the MeCP2³⁰⁸ protein itself

Some embodiments relate to the use of pivagabine to modulate the activity of CRH-producing neurons in patients suffering from RTT.

Pivagabine was initially synthesized in 1978 as an analog of GABA and was intended for use as an antiepileptic drug (AED). Animal and human studies have since confirmed that pivagabine does indeed bear potential as an AED. Pivagabine was initially developed by Angelini Recherche (Rome, Italy) and brought to the market in Italy for the treatment of disorders associated with stress. Several nonclinical studies have shown that pivagabine, particularly under stressful challenges, may modulate the effects of CRH in the rat brain.

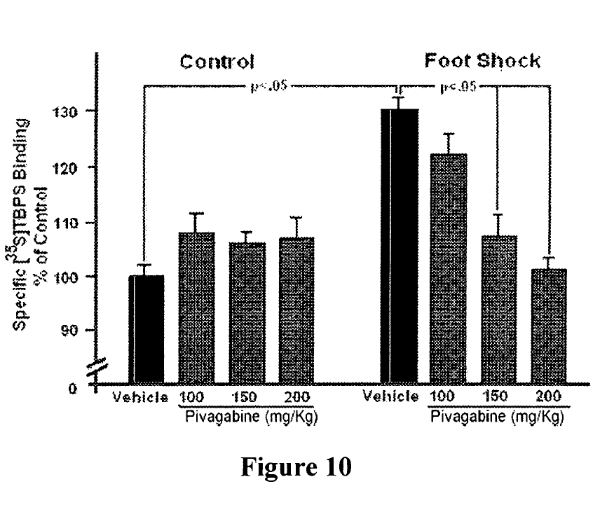

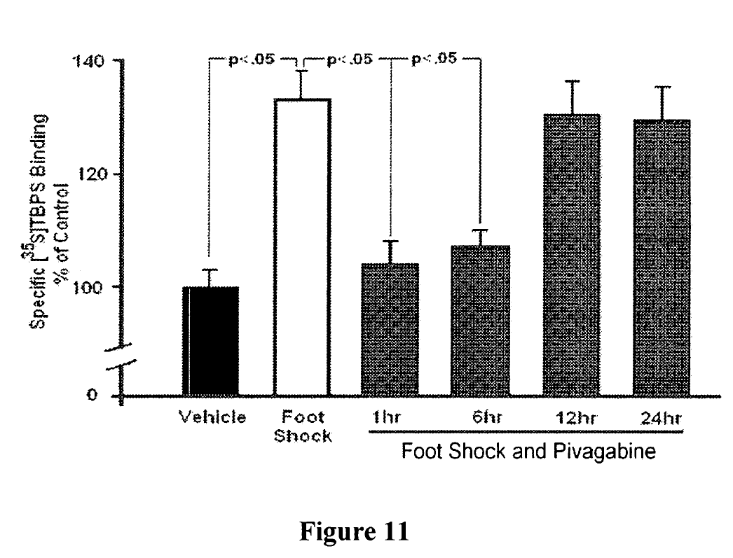

Various acute and subchronic stress paradigms produce a rapid decrease in the function of GABA receptors, as assessed by an increase in t-[35S]butylbicyclo-phosphorothionate ([35S]TBPS) binding, in several regions of the rat brain. Some embodiments relate to the finding shown in FIG. 10 that subchronic treatment with pivagabine (100-200 mg/Kg) results in a dose-dependent inhibition of the foot shock-induced increase in [35S]TBPS binding in cerebral cortical membranes. Other embodiments related to the finding shown in FIG. 11 that the time course of this effect of pivagabine at a dose of 200 mg/Kg shows that the antagonism is significant 1 and 6 hours after the last injection, but is no longer apparent after 12 or 24 hours. Thus, the effects observed follow the pharmacokinetic half-life of pivagabine.

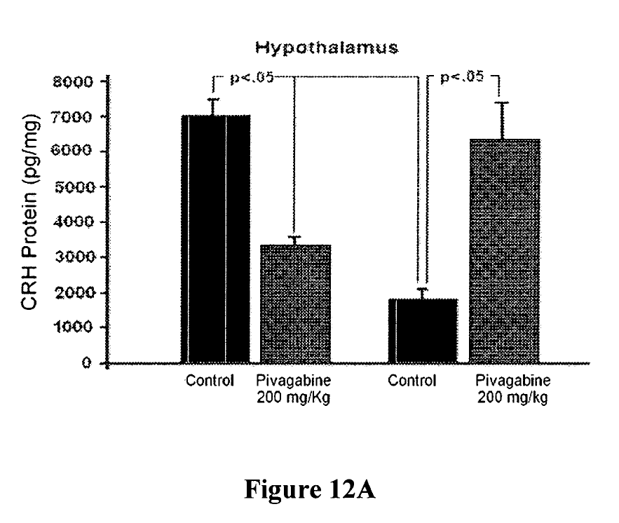

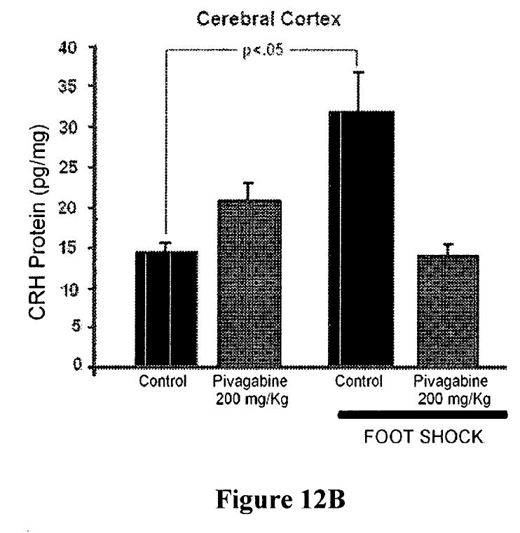

As mentioned above, stress is known to be associated with release of CRH from the hypothalamus and activation of the HPA axis. This is paralleled by changes in the CRH content of different brain regions and alterations in the expression of messenger RNA (mRNA) for CRH. Some embodiments relate to the finding shown in FIGS. 12A and 12B that acute foot-shock stress reduced CRH concentrations in the rat hypothalamus by 74% and increased CRH concentrations in the cerebral cortex by 125%, respectively. Subchronic treatment with pivagabine prevented the effects of foot-shock stress on CRH concentration in both brain regions. Pivagabine alone reduced the CRH concentration in the hypothalamus by 52%, but had no effect in the cerebral cortex. Intra-cerebroventricular administration of CRH to rats is not antagonized by sub-chronic treatment with pigavabine suggesting that pigavabine is not acting as a simple antagonist of CRH.

Studies show that sustained pivagabine treatment increases the abundance of CRH mRNA in both the hypothalamus and cerebral cortex of rats. This effect of the drug in the hypothalamus likely reflects a compensatory response to the associated decrease in the amount of CRH peptide. Therefore, in a stressful condition less CRH will be available to be released, reducing the responses to stress. Thus, both pivagabine and foot-shock stress decreased the hypothalamic content of CRH but with different mechanisms. While the decreased hypothalamic content of CRH after foot-shock stress is immediate, the pivagabine-induced decrease requires repeated administration to lead to increased CRH turnover or decreased availability, as suggested by the increases in its mRNA levels

The present embodiments also include pharmaceutical formulations comprising a therapeutically effective amount of any of the compounds described above and a pharmaceutically acceptable carrier. In one embodiment a pharmaceutical formulation is made by combining any of the compounds described above and a pharmaceutically acceptable carrier. The present embodiments further include a process for making a pharmaceutical formulation comprising combining any of the compounds described above and a pharmaceutically acceptable carrier.

As used herein, the term “pharmaceutically acceptable carrier” includes any and all solvents, dispersion media, coatings, permeation enhancers, antibacterial and antifungal agents, isotonic and absorption delaying agents and the like compatible with pharmaceutical administration.

As used herein, the term “formulation” encompasses a product comprising the specified ingredients in the specified amounts, as well as any product which results, directly or indirectly, from combination of the specified ingredients in the specified amounts.

The present embodiments include pharmaceutical formulations comprising one or more compounds described throughout in association with a pharmaceutically acceptable carrier. Preferably, these formulations are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual, buccal, topical or rectal administration, or for administration by inhalation or insufflation. Also, the instant compounds can be administered to the body through Xenoport technology. XenoPort identifies and characterizes transporters throughout the body that are useful to drug delivery, then uses selected transporter proteins as “targets” and employs medicinal chemistry techniques to modify drugs into substrates for these transporters.

Alternatively, the formulations may be presented in a form suitable for once-daily, once-weekly or once-monthly administration; for example, an insoluble salt of the active compound may be adapted to provide a preparation for intramuscular injection. The pharmaceutical formulations described herein can be administered to a patient per se, or in pharmaceutical formulations where they are mixed with other active ingredients, as in combination therapy, or suitable pharmaceutically acceptable carriers or excipient(s). Techniques for formulation and administration of the compounds of the instant application may be found in “Remington's Pharmaceutical Sciences,” Mack Publishing Co., Easton, Pa., 18th edition, 1990.

For preparing solid pharmaceutical formulations such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation formulation containing a homogeneous mixture of a compound of the present embodiments, or a pharmaceutically acceptable salt thereof. When referring to these preformulation formulations as homogeneous, it is meant that the active ingredient is dispersed evenly throughout the formulation so that the formulation may be readily subdivided into equally effective unit dosage forms such as tablets, pills and capsules. This solid preformulation formulation is then subdivided into unit dosage forms of the type described above containing from about 10 to about 10,000 mg of pivagabine. Preferably the dosage is from about 50 to about 5000 mg; more preferably, the dosage is from about 100 to about 3500 mg; even more preferably, the dosage is from about 450 to about 2400 mg. The tablets or pills of the novel formulation can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Furthermore, compounds for the present embodiments can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

Pharmaceutical formulations for parenteral administration, e.g. by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g. in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use.

For oral administration, the instant compounds can be formulated readily by combining the active compounds with pharmaceutically acceptable carriers well known in the art. Such pharmaceutically acceptable carriers enable the compounds of the present embodiments to be formulated as tablets, pills, dragees, capsules, liquids, gels, syrups, slurries, suspensions and the like, for oral ingestion by a patient to be treated. Pharmaceutical formulations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal administration, the pharmaceutical formulations may take the form of tablets, lozenges, wafers and rapid-dissolve preparations formulated in conventional manner.

The compounds of the present embodiments can also be administered in the form of liposome pharmaceutical formulations, such as small unilamellar vesicles, large unilamellar vesicles and multilamellar vesicles. Liposomes can be formed from a variety of phospholipids, such as cholesterol, stearylamine or phosphatidylcholines.

Further disclosed herein are various pharmaceutical formulations well known in the pharmaceutical art for uses that include intraocular, intranasal, and intraauricular delivery. Suitable penetrants for these uses are generally known in the art. Pharmaceutical formulations for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts.

Alternatively, other delivery systems for hydrophobic pharmaceutical compounds may be employed. Liposomes and emulsions are well known examples of delivery vehicles or pharmaceutical acceptable carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The dosage regimen utilizing the compounds of the present embodiments is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the pharmaceutical formulation's availability to target sites. This involves a consideration of the distribution, equilibrium, and elimination of the compounds. Advantageously, compounds of the present embodiments may be administered, for example, in a single daily dose, or the total daily dosage may be administered in divided doses of two, three or four times daily.

In the methods of the present embodiments, the pharmaceutical formulations herein described in detail are typically administered in accordance with conventional pharmaceutical practices.

For example, for oral administration in the form of a tablet or capsule, the compounds of the present embodiments can be combined with an oral, non-toxic pharmaceutically acceptable inert carrier such as ethanol, glycerol, water and the like. Moreover, when desired or necessary, suitable pharmaceutically acceptable carriers, such as, binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Some examples of pharmaceutically acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety.

The oral liquid formulations in which the present embodiments may be incorporated for administration orally include using pharmaceutically acceptable carriers, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous oral suspensions include synthetic and natural gums such as tragacanth, acacia, alginate, dextran, sodium carboxymethylcellulose, methylcellulose, polyvinyl-pyrrolidone or gelatin. Other dispersing agents which may be employed include glycerin and the like.

The daily dosage of the products may be varied over a wide range; e.g., from about 10 to about 10,000 mg per adult human per day. For oral administration, the formulations are preferably provided in the form of tablets containing about 1.0, 10.0, 15.0, 25.0, 50.0, 100, 200, 300, 400, 500, 600, 700, 800, 900 1000, 2000, 3000, 4000, 5000, 6000, 7000, 8000, 9000 or 10,000 milligrams of the active ingredient for the symptomatic adjustment of the dosage to the patient to be treated. The instant pharmaceutical formulations typically contain from 10 mg to about 2000 mg of the instant compounds, preferably, from about 50 mg to about 1000 mg of active ingredient. An effective amount of the instant compounds is ordinarily supplied at a dosage level of from about 0.002 mg/kg to about 150 mg/kg of body weight per day. Preferably, the range is from about 0.02 to about 80 mg/kg of body weight per day, and especially from about 0.2 mg/kg to about 40 mg/kg of body weight per day. The compounds may be administered on a regimen of about 1 to about 10 times per day.

As used herein, “Central Nervous System disorder” refers to any neurological disorder that affects the brain or spinal column, including, but not limited to, acute stress disorder; affective disorders, including depressive disorders (major depressive disorder, dysthymia, childhood depression, atypical depression, bipolar disorder, mania and hypomania) and anxiety disorders (generalized anxiety disorder, social anxiety disorder, phobias, obsessive compulsive disorder, panic disorder, post-traumatic stress disorder); premenstrual dysphoric disorder (also known as pre-menstrual syndrome); psychotic disorders, such as brief psychotic disorder, schizophrenia, psychotic mood disorder (depression and/or mania); attention deficit disorder (with and without hyperactivity); obesity, eating disorders such as anorexia nervosa and bulimia nervosa; vasomotor flushing; cocaine and alcohol addiction; sexual dysfunction and related illnesses; acute and chronic pain syndromes, as exemplified by fibromyalgia, neurasthenia, chronic low back pain, trigeminal neuralgia; visceral pain syndromes, such as irritable bowel syndrome, noncardiac chest pain, functional dyspepsia, interstitial cystitis, essential vulvodynia, urethral syndrome, orchialgia, temperomandibular disorder, atypical face pain, migraine headache, and tension headache; functional somatic disorders, for example, chronic fatigue syndrome; neurologic disorders including seizure disorder, Tourette Syndrome, Parkinson's Disease, Huntington's Chorea, Alzheimer's Disease, subcortical and other dementias, Tardive Dyskinesia, Multiple Sclerosis, Rett Syndrome or amyotrophic lateral sclerosis.

As used herein, the term “patient” refers to the recipient of a therapeutic treatment and includes all organisms within the kingdom animalia. In preferred embodiments, the animal is within the family of mammals, such as humans, bovine, ovine, porcine, feline, buffalo, canine, goat, equine, donkey, deer and primates. The most preferred animal is human.

As used herein, the terms “treat” “treating” and “treatment” include “prevent” “preventing” and “prevention” respectively.

The instant compounds may be synthesized by methods described above, or by modification of these methods. Ways of modifying the methodology include, among others, temperature, solvent, reagents etc., and will be obvious to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and Greene & Wuts, Protective Groups in Organic Synthesis, John Wiley & Sons, 1991, which are both hereby incorporated herein by reference in their entirety. The protecting groups may be removed at a convenient subsequent stage using methods known in the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety.

Where the processes for the preparation of the compounds disclosed herein give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid, followed by fractional crystallization and regeneration of the free base. The compounds may also be resolved using a chiral auxiliary by formation of diastereomeric derivatives such as esters, amides or ketals followed by chromatographic separation and removal of the chiral auxiliary.

As used herein, “treat, treating and treatment” of a subject includes the application or administration of a formulation of the present embodiments to a subject, or application or administration of a formulation of the present embodiments to a cell or tissue from a subject, who has a central nervous system disease, disorder or condition, has a symptom of such a disease, disorder or condition, or is at risk of (or susceptible to) such a disease, disorder or condition, with the purpose of curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of disease. The treatment or amelioration of symptoms can be based on objective or subjective parameters; including the results of a physical examination, a psychiatric evaluation, including, for example, symptom ratings, such as the Clinical Dementia Rating (CDR), Mini-Mental State Examination (MMSE), Alzheimer Disease Assessment Scale-Cognitive (ADAS-Cog); a laboratory test indicating perturbations of the stress axis, such as dexamethasone suppression test (DST), the CRF challenge test; or another test known in the art. For example, the instant compounds, methods and formulations provide the treatment and prevention of diseases or disorders involving perturbation of the biological stress mechanisms including, but not limited to, acute stress disorder; affective disorders, including depressive disorders (major depressive disorder, dysthymia, childhood depression, atypical depression, bipolar disorder, mania and hypomania) and anxiety disorders (generalized anxiety disorder, social anxiety disorder, phobias, obsessive compulsive disorder, panic disorder, post-traumatic stress disorder); premenstrual dysphoric disorder (also known as pre-menstrual syndrome); psychotic disorders, such as brief psychotic disorder, schizophrenia, psychotic mood disorder (depression and/or mania); attention deficit disorder (with and without hyperactivity); obesity, eating disorders such as anorexia nervosa and bulimia nervosa; vasomotor flushing; cocaine and alcohol addiction; sexual dysfunction and related illnesses; acute and chronic pain syndromes, as exemplified by fibromyalgia, chronic low back pain, trigeminal neuralgia; visceral pain syndromes, such as irritable bowel syndrome, noncardiac chest pain, functional dyspepsia, interstitial cystitis, essential vulvodynia, urethral syndrome, orchialgia, temperomandibular disorder, atypical face pain, migraine headache, and tension headache; functional somatic disorders, for example, chronic fatigue syndrome; neurologic disorders including seizure disorder, Tourette Syndrome, Parkinson's Disease, Huntington's Chorea, Alzheimer's Disease, subcortical and other dementias, Tardive Dyskinesia, Multiple Sclerosis, Rett Syndrome or amyotrophic lateral sclerosis.

The instant compounds can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. For example, efficacy for depressive and anxiety or stress disorders can be predicted from animal models that include, but are not limited to, the Vogel Conflict Test, the Forced Swim Test, the Tail Suspension Test; efficacy for acute and chronic pain conditions can be predicted from animal models exemplified by the Tail Flick Test, the Hot Plate Test, Active and Passive Mechanical or Thermal Allodynia Tests, nerve compression or section test. When selecting a model to determine efficacy, the skilled artisan can be guided by the state of the art to choose an appropriate model, dose, and route of administration, and regime. Of course, human clinical trials can also be used to determine the efficacy of a compound in humans. The instant compounds and formulations can be screened using a combination of in vitro and in vivo techniques. The in vitro testing can involve measuring properties such as solubility, logP, permeability across membranes, and susceptibility to hydrolysis by esterases in the blood. In vivo testing can assess plasma-to-brain ratios of the concentration of pivagabine.

In one exemplary embodiment, an 70 kg adult patient with RTT or displaying symptoms associated with RTT is given 1 mg to 10 g of pivagabine once daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, an 70 kg adult patient with RTT or displaying symptoms associated with RTT is given 1 mg to 10 g of pivagabine twice daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 10 mg to 8000 mg of pivagabine once daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 10 mg to 8000 mg of pivagabine twice daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 100 mg to 6000 mg of pivagabine once daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 100 mg to 6000 mg of pivagabine twice daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 250 mg to 4000 mg of pivagabine once daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 250 mg to 4000 mg of pivagabine twice daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 350 mg to 3500 mg of pivagabine once daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 350 mg to 3500 mg of pivagabine twice daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 450 mg to 2400 mg of pivagabine once daily to treat RTT or the symptoms associated with RTT. This dosage can be adjusted based on the results of the treatment and the judgment of the attending physician. Treatment is preferably continued for at least about 1 or 2 weeks, preferably at least about 1 or 2 months, and may be continued on a chronic basis.

In one exemplary embodiment, a 70 kg adult patient with RTT or displaying symptoms associated with RTT is given 450 mg to 2400 mg of pivagabine twice daily to treat

The following examples are provided for illustrative purposes only, and are in no way intended to limit the scope of the present embodiments.

### EXAMPLE 1

### Electroshock-Induced Convulsions in Rats

Male rats were studied using the method described by E. Chojnacka-Wojcik (Pol. J. Pharmacol. Pharm., 35:511-15, 1983), herein incorporated by reference in its entirety. Animals were randomized to receive either vehicle, pivagabine (100 or 200 mg/Kg i.p.) or luminal sodium (10 mg/Kg i.p.).

At 30 minutes after dosing, the animals were given a non-lethal electric shock (50 Hz, 50 mA, impulse train duration 0.2 s via corneal electrodes wetted in normal saline). Animals were observed for tonic and/or clonic convulsions. The frequency of tonic and clonic convulsions among the treatment groups was analyzed using the Yates chi-square test .

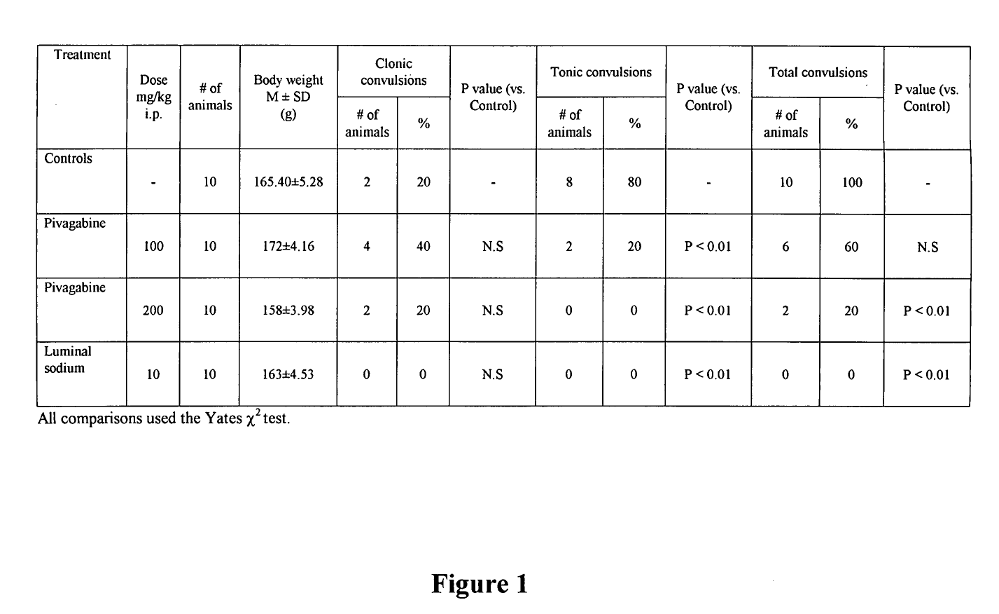

Eight of ten electroshock-induced animals showed tonic convulsions while two showed clonic convulsions. Luminal sodium was used as a positive control which completely blocked tonic and clonic convulsions. The results of the test are summarized in FIG. 1 and demonstrate that treatment of rats with pivagabine significantly reduced the number of tonic convulsions in response to electric shock.

### EXAMPLE 2

### Convulsions and Strychnine-Induced Mortality in Mice

Strychnine causes muscular convulsions and eventual death through asphyxia or sheer exhaustion in mammals. The endpoint of the test, as described in Kerley T. L. et al. J. Pharmacol. Test Ther. 132:360; 1961, herein incorporated by reference, was an increase in the lethal dose level of strychnine in animals treated with pivagabine as compared to control or the reference compound, mephenesine.

Three groups of ten male mice weighing 21-24 g were randomized to receive, pivagabine (100 mg/Kg) or mephenesine (100 mg/Kg) via intra peritoneal (i.p.) administration 30 minutes prior to administration of strychnine. Strychnine was administered i.p., dissolved in normal saline at a constant volume of 10 ml/kg. An initial dose of strychnine 1.3 mg/kg i.p. was administered in each treatment, followed by lower doses until a 10-20% mortality rate was obtained, and then by higher doses until a 70-90% mortality rate was obtained. The number of deaths was recorded, starting 15 minutes after administration. Using data on deaths at each dose, the LD₅₀, the 95% confidence intervals, and the line slope S were calculated.

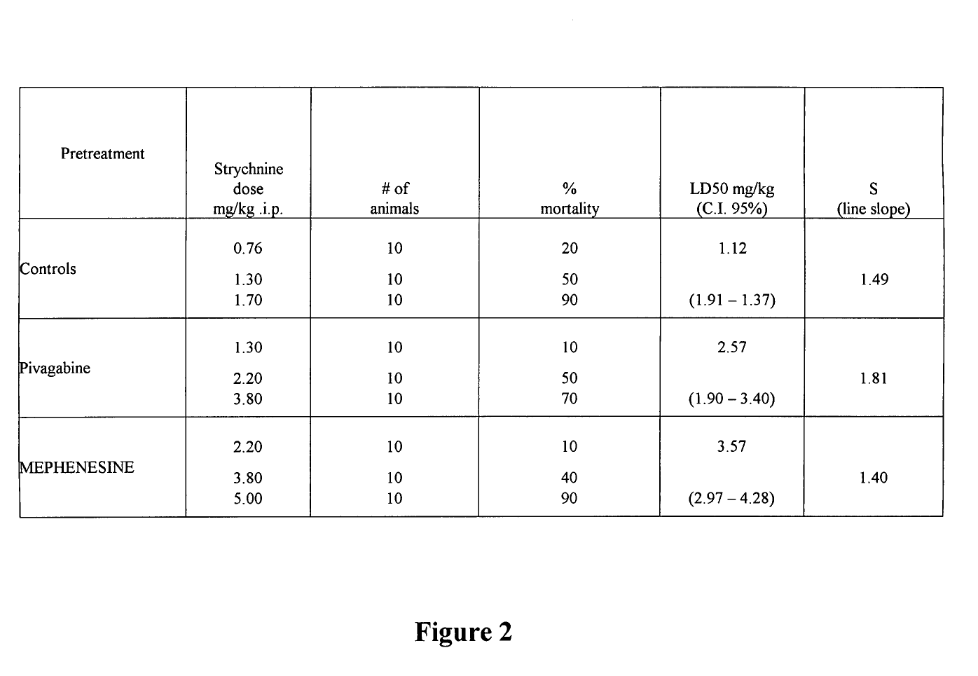

As shown in FIG. 2, both mephenesine and pivagabine raised the LD50 of strychnine, suggesting that pivagabine is capable of protecting an animal from strychnine-induced death. However, the slope of the dose/mortality line for mephenesine-pretreated animals runs parallel to that of the controls indicating a competitive antagonism of strychnine. In contrast, the slope of the line representing the pivagabine-pretreated animals shows a very different slope, indicating a nonspecific antagonism of the effects of strychnine.

### EXAMPLE 3

### Pentetrazol-Induced Convulsions in Rats

The method proposed by Goodman L. S. et al. (J. Pharmacol. Test Ther. 108:168; 1958) was used. Six groups of 10 female rats each were randomized to receive either vehicle, pivagabine (50 or 100 mg/kg i.p.), luminal sodium (10 mg/kg i.p.) or GABA (50 or 100 mg/kg i.p.). After 30 minutes, pentylene tetrazol (80 mg/kg i.p.) was administered. The number of animals from each group who manifested at least one convulsion during the 30 minutes after injection was counted. The differences between treatments were statistically analyzed using the chi-square/Fisher exact test.

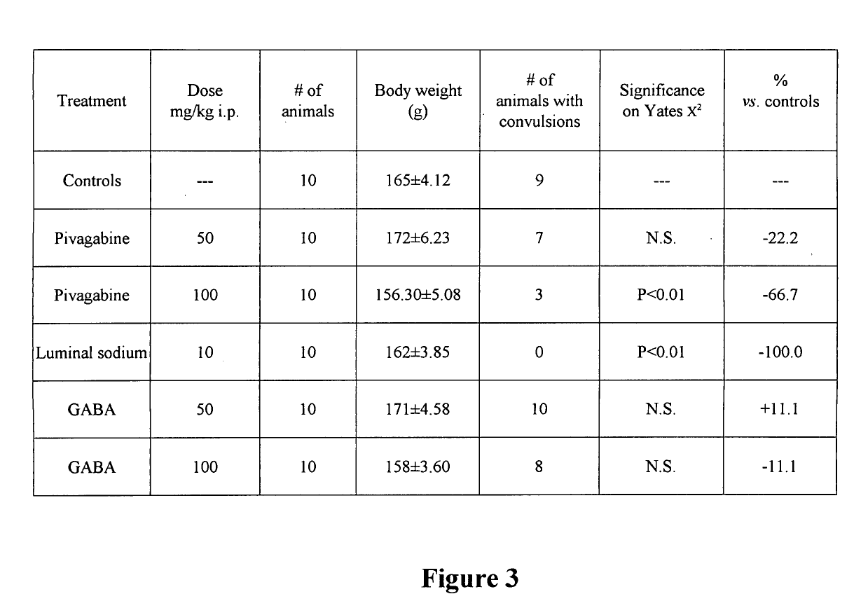

As shown in FIG. 3, 90% of the animals in the control group manifested clonic or tonic convulsions after the administration of pentetrazol 80 mg/kg i.p. Only 30% of the treated animals manifested convulsions under pretreatment with pivagabine 100 mg/kg (p<0.01). None of the animals treated with luminal sodium 10 mg/kg i.p. manifested convulsions of any kind. GABA showed no anticonvulsant effect at either dose.

### EXAMPLE 4

### Bicucullin-Induced Convulsions in Rats

The method described by Curtis D. R. et al. (Brain Res. 34:301; 1971), herein incorporated by reference in its entirety, was used in modified form. Six groups of ten each fasted male rats were randomized to receive oral doses of either vehicle, pivagabine (100 or 200 mg/kg), luminal sodium (20 mg/kg) or GABA (100 and 200 mg/kg).

One hour later, bicuculline was administered by intraperitoneal route at a dose of 4.5 mg/kg dissolved in 10 ml/kg NSS. The number of animals manifesting any type of convulsion was then counted. The protection conferred by the treatments was then calculated as percent compared with the controls. The statistical significance of the differences was analyzed using the Yates chi-square test.

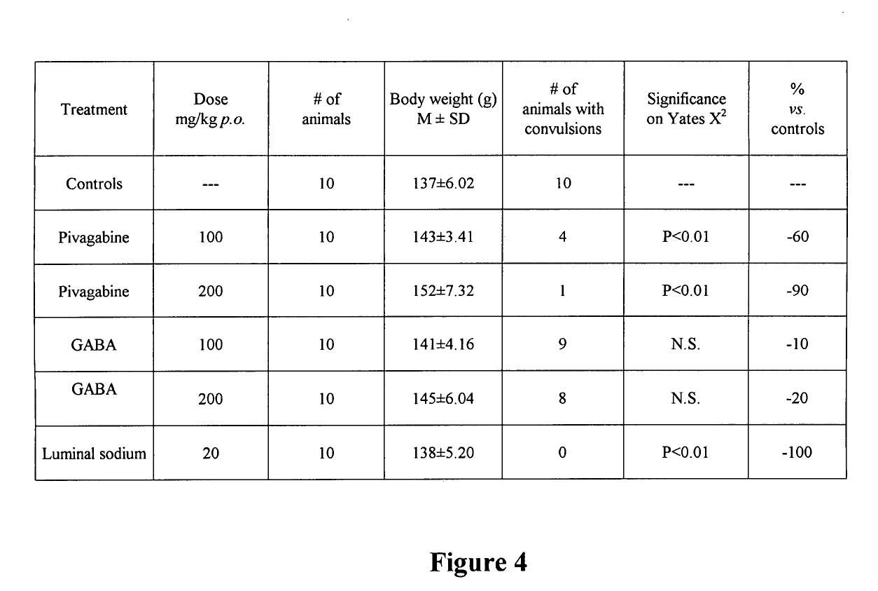

The results are summarized in FIG. 4 and show that all of the animals in the control group manifested tonic or clonic convulsions. Animals pre-treated with pivagabine at doses of 100 or 200 mg/kg orally showed convulsions in 4/10 and 1/10 animals for each dose respectively. Thus, protection against convulsions was 90% at the higher dose. GABA was barely active at either of the doses used, providing 10% and 20% protection at the low and high dose respectively. Luminal sodium provided full 100% protection.

### EXAMPLE 5

### Isoniazide-Induced Convulsions in Rats

The method of Reilly R. H. et al. (JAMA 152(14):1317; 1953), herein incorporated by reference in its entirety, was used in modified form. The objective of the first part of the test was to examine the protective effect provided by pivagabine (at oral doses of 25-50-100-200 mg/Kg) administered 1 hour prior to treatment with the convulsive agent isoniazide (100 mg/kg i.p.). Both the ED50 and ED84 were calculated. In the second part of the test, the ED84 was administered at different times (0.5-1.0-1.5-2.0-3.0 hours) prior to isoniazide administration for the purpose of determining the duration of action and the curve of kinetic activity. Female rats fasted for 17 hours were used for the test.

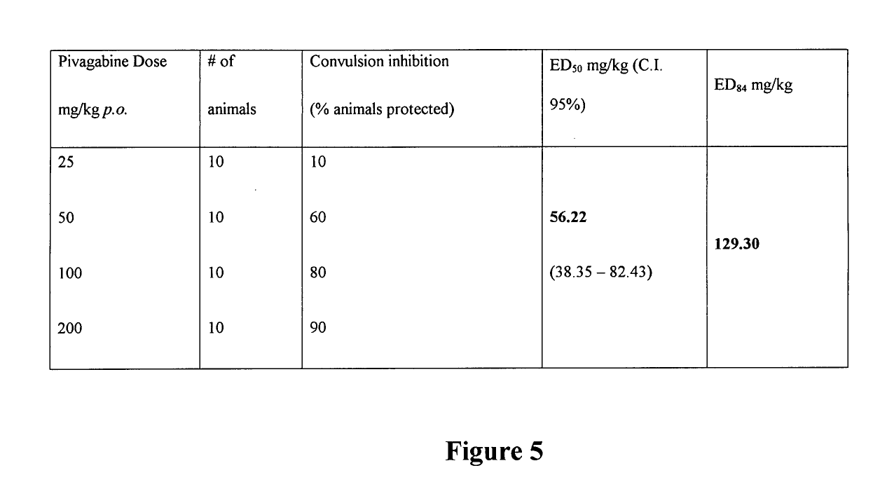

As shown in FIG. 5, a dose-related increase in anticonvulsive effect was seen with pivagabine. The highest dose provided 90% protection against seizures.

Dose-effect relationship of pivagabine administered 1 hour prior to isoniazide (100 mg/kg i.p.).

### EXAMPLE 6

### Activity in an Ethological Test—Open Field Behavior

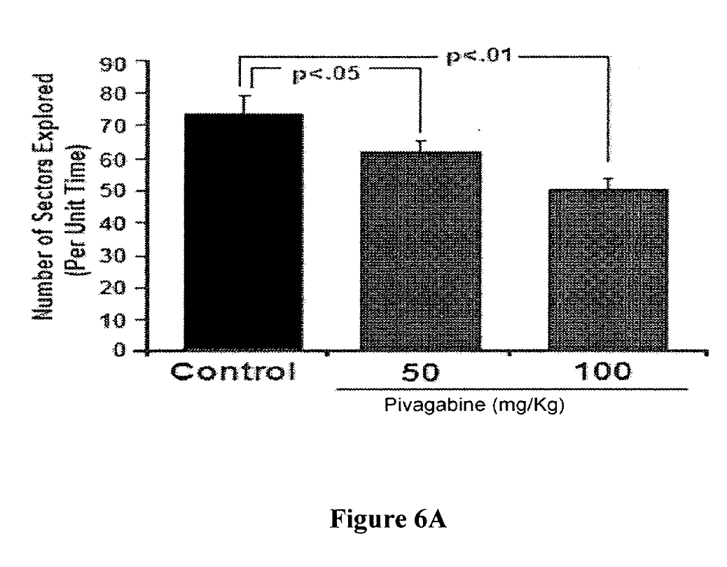

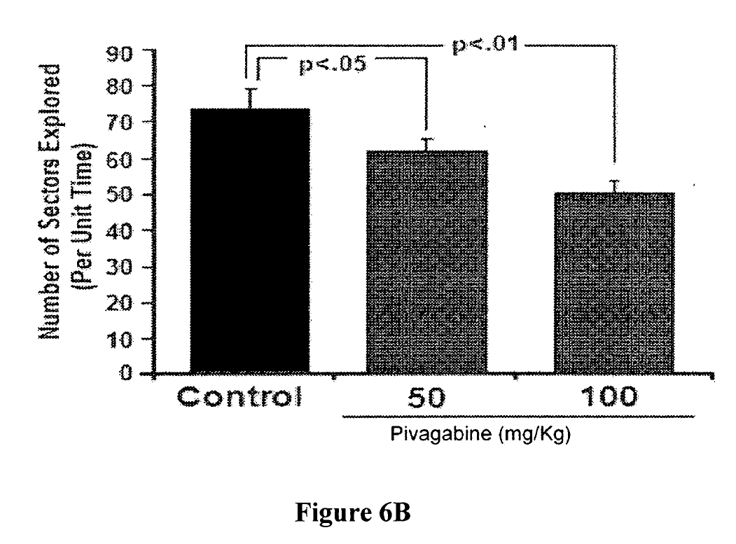

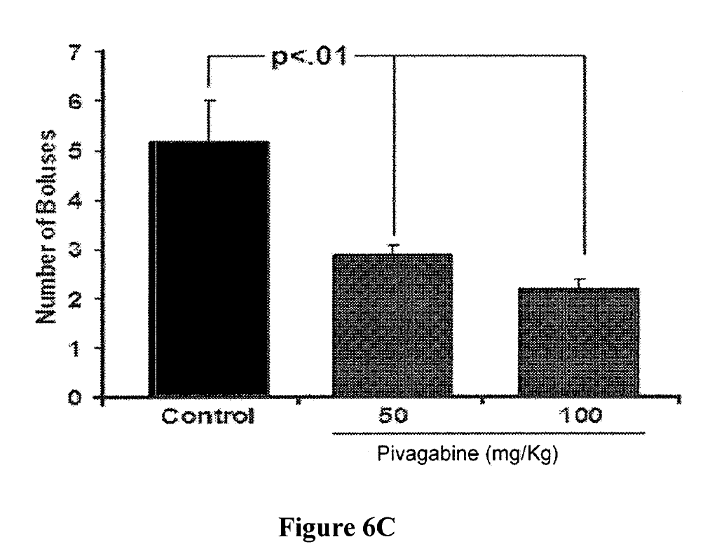

The open-field behavior test relies on the observation that rats exposed to a novel open environment display behaviors (exploratory ambulation, rearing, and dropping fecal boluses) that are thought to be naturally defensive and anticipatory of potential threats in the environment. Pharmacologic interventions that attenuate these behaviors may have utility in modifying pathologic fear or stress responses in a clinical context. FIG. 6A demonstrates a significant decrease in ambulation as measured by the number of sectors explored per unit time. FIG. 6B demonstrates that pivagabine dosed at 50 and 100 mg/Kg i.p. significantly reduced the number of rearing movements. FIG. 6C demonstrates a significant decrease in defecation, as measured by the number of fecal boluses produced.

### EXAMPLE 7

### Water Maze Test

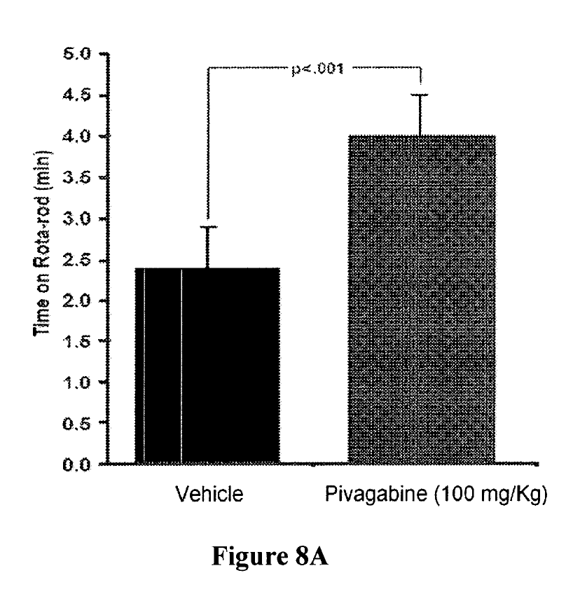

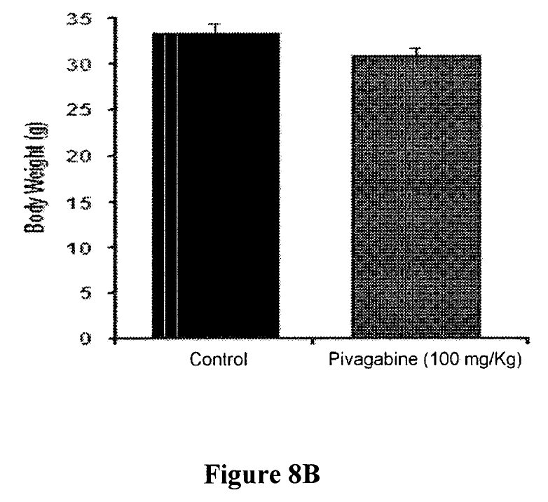

The water maze test is designed to test the effect of drugs on learning and performance in a task that is performed under a stressful condition. After several trials under control conditions, rats learn to exit the water maze with greater speed and with fewer errors. Performance in this test can be improved by drugs that either reduce stress and/or enhance learning (e.g. muscarinic agonists). On the contrary, drugs that impair learning (e.g. scopolamine) can worsen performance. Rats treated with pivagabine 100 mg/Kg performed as well as the control group, in terms of the time to exit (FIG. 8A) and the number of errors made (FIG. 8B). In later sessions (days 4 and 5), the performance of pivagabine treated animals tended to be superior to that of controls. This indicates improved learning and retention.

### EXAMPLE 8

### Rota-Rod Test

Motor performance can be tested by measuring the length of time an animal can remain on a slowly rotating cylinder, as in the rota-rod test. FIG. 8A shows that mice treated with pivagabine 100 mg/Kg remained on the rotating cylinder significantly longer than untreated mice. FIG. 8B demonstrates that the differences in time were not accounted for by body weight. As such, mice treated with pivagabine displayed heightened motor performance relative to untreated mice.

### EXAMPLE 9

### Taut Thread Test

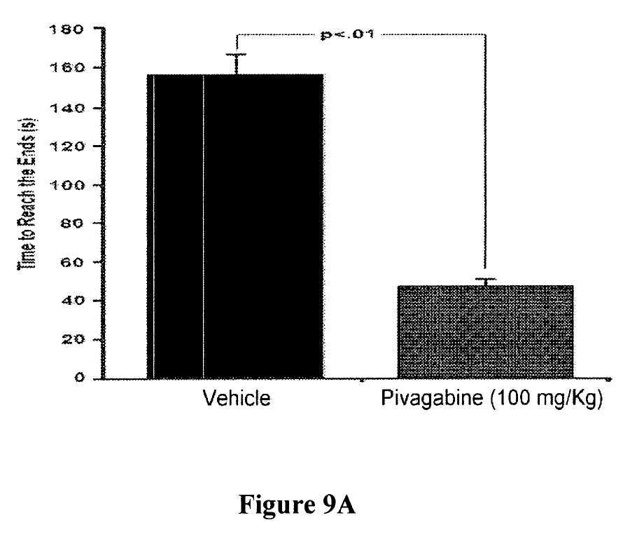

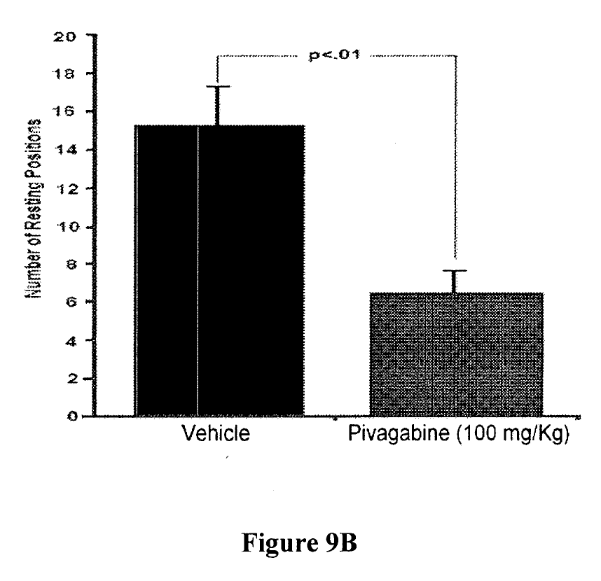

In this test, aged mice are required to traverse a thread stretched between two points. Performance is measured by the time taken to reach the end and the number of resting positions adopted during the test. FIG. 9A demonstrates that mice treated with pivagabine traversed the thread significantly faster than control mice. Additionally, FIG. 9B demonstrates that mice treated with pivagabine adopted significantly less resting positions during the test.

### Equivalents

The foregoing written specification is considered to be sufficient to enable one skilled in the art to practice the invention. The foregoing description details certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method of treating a patient with symptoms of Rett Syndrome, comprising administering an amount of pivagabine to the patient effective to reduce said symptoms.

2. The method of claim 1 wherein the patient is a mammal.

3. The method of claim 1, wherein the patient is a human.

4. The method of claim 1, wherein the symptoms are selected from anxiety, ataxia, stressful reactions to novel situations, convulsions, compulsive or repetitive behavior, chorea, memory impairment, social withdrawal, and mental retardation.

5. The method of claim 1, wherein the pivagabine is administered by one or more of the routes consisting of intravenous, intraperitoneal, inhalation, intramuscular, subcutaneous and oral.

6. The method of claim 1, wherein the pivagabine is administered orally.

7. The method of claim 1, wherein the pivagabine has the following structure:

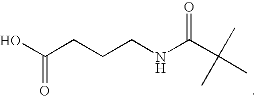

8. A method of treating the indications of Rett Syndrome in a patient comprising administering to said patient a therapeutically effective amount of pivagabine.

9. The method of claim 8, wherein the patient is a mammal.

10. The method of claim 8, wherein the patient is a human.

11. The method of claim 8, wherein the pivagabine is administered by one or more of the routes consisting of intravenous, intraperitoneal, inhalation, intramuscular, subcutaneous and oral.

12. The method of claim 8, wherein the pivagabine is administered orally.

13. The method of claim 8, wherein the pivagabine has the following structure:

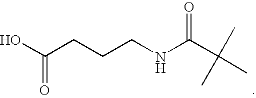

14. A method of treating or preventing the symptoms of Rett Syndrome in a mammal, comprising the step of administering to said mammal a therapeutically effective amount of pivagabine and a pharmaceutically acceptable vehicle.

15. The method of claim 14, wherein the mammal is human.

16. The method of claim 14, wherein the symptoms are selected from anxiety, ataxia, stressful reactions to novel situations, convulsions, compulsive or repetitive behavior, chorea, memory impairment, social withdrawal, and mental retardation.

17. The method of claim 14, wherein the pivagabine is administered by one or more of the routes consisting of intravenous, intraperitoneal, inhalation, intramuscular, subcutaneous and oral.

18. The method of claim 14, wherein the pivagabine is administered orally.

19. The method of claim 14, wherein the pivagabine has the following structure:

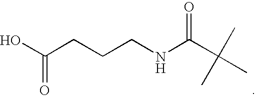

20. A method of treating Rett Syndrome, comprising administering pivagabine.

21. The method of claim 20, wherein the pivagabine is administered by one or more of the routes consisting of intravenous, intraperitoneal, inhalation, intramuscular, subcutaneous and oral.

22. The method of claim 20, wherein the pivagabine is administered orally.

23. The method of claim 20, wherein the pivagabine has the following structure: [chemical structure]
